(19) United States
(12) Patent Application Publication
(10) Pub. No.: US 2017/0135646 A1
(43) Pub. Date: May 18, 2017

(54) HYPERSPECTRAL IMAGING FOR PREDICTION OF SKIN INJURY AFTER EXPOSURE TO THERMAL ENERGY OR IONIZING RADIATION

(71) Applicant: UNIVERSITY OF MASSACHUSETTS, Boston, MA (US)

(72) Inventor: Michael S. Chin, Worcester, MA (US)

(21) Appl. No.: 15/311,589

(22) PCT Filed: May 29, 2015
(86) PCT No.: PCT/US15/33229

§ 371 Date: Nov. 16, 2016

Related U.S. Application Data
(60) Provisional application No. 62/007,891, filed on Jun. 4, 2014.

(51) Int. Cl. A61B 5/00; A61B 5/1455

(52) U.S. Cl. CPC A61B 5/7275; A61B 5/0075; A61B 5/14551; A61B 5/445; A61B 5/443; A61B 2576/00

(57) ABSTRACT

The invention provides hyperspectral imaging-based methods that enable effective, efficient and non-invasive detection and characterization of thermal and ionizing radiation exposure in tissue. The methods allow for complete visualization and quantification of oxygenation and perfusion changes in thermal burn or ionizing radiation impacted skin and enables rapid identification of individuals exposed to such exposures and allows early prediction of extent of injury in normal tissue after exposure.

### PRIORITY CLAIMS AND RELATED PATENT APPLICATIONS

This application claims the benefit of priority from U.S. Provisional Application Ser. No. 62/007,891, filed on Jun. 4, 2014, the entire content of which is incorporated herein by reference in its entirety.

### TECHNICAL FIELDS OF THE INVENTION

The invention generally relates to methods for detecting and analyzing exposure of tissue to thermal burn or ionizing radiation. More particularly, the invention relates to methods for characterization, evaluation and prediction of injuries from thermal or radiation exposures in tissue and their effect thereof using hyperspectral imaging-based techniques.

### BACKGROUND OF THE INVENTION

Exposure to thermal burn or ionizing radiation can have profound biological consequences to skin and underlying subcutaneous tissue. Skin structures, especially the dermal plexus, demonstrate changes in response to both thermal and ionizing radiation energy. After exposure to thermal energy, depth of burn injury predicts the healing of the burn wound, and thus early and accurate assessment is of great importance in the care of the burn patient.

Thermal injury is divided into four degrees of depth or severity: superficial, superficial-partial, deep-partial, and full thickness burns. For full thickness and deep dermal burns, early excision and skin grafting of full thickness and deep dermal burns has been shown to be therapeutically and financially advantageous, with shorter healing time, fewer infections, better functional and aesthetic results, as well as reduced hospital stay and lower treatment cost. (Feller, et al. 1980 JAMA 244.18: 2074-2078; Herndon, et al. 1989 Annals of Surgery 209.5: 547; Ong, et al. 2006 Burns 32.2: 145-150; Heimbach, et al. 1992 World Journal of Surgery 16.1: 10-15; Prasanna, et al. 1994 Burns 20.5: 446-450; Burke, et al. 1976 The Surgical clinics of North America 56.2: 477-494.)

Early excision and skin grafting of full-thickness and deep dermal burns has been shown to be therapeutically and financially advantageous, with shorter healing time, fewer infections, better functional and aesthetic results, as well as reduced hospital stay. However, early differentiation of superficial dermal versus deep dermal burns presents a diagnostic challenge. Superficial and intermediate dermal burns heal with conservative management within two to three weeks. However, deep dermal burns result in prolonged hospitalizations, increased risk of infection, excessive scarring and thus are best managed by excision and skin grafting. (Johnson, et al. 2003 Advances in Skin & Wound Care 16.4: 178-187; Jaskille, et al. 2009 Journal of Burn Care & Research 30.6: 937-947.) Differentiation between dermal burns that spontaneously heal versus those that require excision remains clinically challenging, with only 50-75% accuracy in early clinical assessments. (Heimbach, et al. 1992 World Journal of Surgery 16.1: 10-15; Alsbjörn, et al. 1984 Scandinavian Journal of Plastic and Reconstructive Surgery and Hand Surgery 18.1: 75-79; Niazi, et al. 1993 Burns 19.6: 485-489; Pape, et al. 2001 Burns 27.3: 233-239; Droog, et al. 2001 Burns 27.6: 561-568.)

Current standard of care involves waiting for progression of burn injury over the course of days until it becomes clinically evident whether spontaneous healing will occur or excision and grafting is required. Basic clinical overestimation of burn depth results in unnecessary surgery, while underestimation results in prolonged hospital stays, increased morbidity and mortality, delayed surgery, and inferior functional and aesthetic outcomes.

Thus, early reliable differentiation between superficial and deep dermal burns remains a top priority in burn research. In addition, current fluid resuscitation formulas are based on visual estimates of total body surface area burned. Over or under-estimation of burn injury results in inappropriate fluid resuscitation of the patient and potentially life threatening complications. (Heimbach, et al. 1992 World Journal of Surgery 16.1: 10-15; Johnson, et al. 2003 Advances in Skin & Wound Care 16.4: 178-187; Jaskille, et al. 2009 Journal of Burn Care & Research 30.6: 937-947; Alsbjörn, et al. 1984 Scandinavian Journal of Plastic and Reconstructive Surgery and Hand Surgery 18.1: 75-79; Niazi, et al. 1993 Burns 19.6: 485-489; Pape, et al. 2001 Burns 27.3: 233-239; Droog, et al. 2001 Burns 27.6: 561-568; Hoeksema, et al. 2009 Burns 35.1: 36-45; Park, et al. Burns (2013) 655-661.)

Among the various modalities to distinguish burns, histology remains the gold standard. Due to iatrogenic injury and sampling error potential, however, it has little application in actual clinical practice. (Heimbach, et al. 1992 World Journal of Surgery 16.1: 10-15; Jaskille, et al. 2009 Journal of Burn Care & Research 30.6: 937-947; Droog, et al. 2001 Burns 27.6: 561-568; Hoeksema, et al. 2009 Burns 35.1: 36-45.).

In addition, exposure to ionizing radiation can also have profound biological consequences to the skin. Scenarios for radiation exposure range from external beam radiotherapy for oncologic treatment to uncontrolled release events such as a nuclear attack or accident. The disasters it the Fukushima and Chernobyl emphasize the real possibility of the latter scenario. Cutaneous skin reactions in response to ionizing radiation exposure are major consequences in both cases. These skin reactions in the acute phase can range from mild skin erythema to ulceration. For radiotherapy alone, it has been reported that a majority of patients receiving radiotherapy experience some degree of skin reaction. (Murray et al. 2011 Radiother Oncol. 99(2):231-4)

With regard to radiation, two significant issues remain unaddressed: (1) the relationship between the dose of cutaneous irradiation and the magnitude of cutaneous perfusion changes observed over time, and (2) the relationship between early cutaneous perfusion changes and the development of acute or chronic skin injury post-irradiation. The latter posses a significant clinical problem since the development of acute skin reactions during radiotherapy usually signal a treatment break. It has been estimated that a treatment break of a more than a week during breast cancer radiotherapy can have significant negative impact on recurrence rate and overall survival. (Bese, et al. 2007 Journal of BUON: official journal of the Balkan Union of Oncology 12(3):353-9. Bese, et al. 2005 Oncology. 69(3):214-23)

The ability to detect and predict high-risk areas for developing skin reactions and change the treatment plan may prevent the need for these detrimental treatment breaks.

Therefore, an urgent need continues to exist for effective, efficient, non-invasive methods for detection and characterization of both thermal burn and ionizing radiation exposure in tissue.

### SUMMARY OF THE INVENTION

The invention is based on the discovery of hyperspectral imaging-based methods that enable effective, efficient and non-invasive detection, characterization and prediction of the effect of thermal and ionizing radiation exposure in tissue. Methods of the invention allow for complete visualization and quantification of oxygenation and perfusion changes in thermal burn or ionizing radiation impacted skin. The invention enables rapid identification of individuals exposed to such exposures and allows early prediction of extent of injury in normal tissue after exposure.

In one aspect, the invention generally relates to a method for predicting a maximal depth of thermal burn injury formation in superficial tissue of a subject. The method includes: acquiring photographic imagery of one or more areas of superficial tissue of the subject at one or more wavelengths of light and one or more time points; and characterizing the obtained photographic imagery to measure one or more physiological properties in the one or more areas of superficial tissue to predict the maximal depth of burn injury formation in superficial tissue of the subject.

In another aspect, the invention generally relates to a biomedical imaging method for predicting acute skin reactions after exposure to ionizing radiation. The method includes: acquiring photographic imagery of one or more areas of superficial tissue of the subject at one or more wavelengths of light and one or more time points; and characterizing the obtained photographic imagery to detect changes in tissue oxygenation and perfusion levels of the subject to predict acute skin reactions.

In yet another aspect, the invention generally relates to a biomedical imaging method for predicting acute skin reactions after exposure to thermal injury. The method includes: acquiring photographic imagery of one or more areas of superficial tissue of the subject at one or more wavelengths of light and one or more time points; and characterizing the obtained photographic imagery to detect changes in tissue oxygenation and perfusion levels of the subject to predict acute skin reactions.

### BRIEF DESCRIPTION OF THE DRAWINGS

FIG. 1: Relative vessel density at 4 weeks after radiation dose demonstrated an inverse linear relationship with initial radiation dose exposure. (r=0.90, p<0.0001)

FIG. 2: Relative deoxyhemoglobin changes decreased over the the first 3 days for each level of radiation exposure. Note the increasing rate of change as the radiation dose becomes larger.

FIG. 3: Deoxyhemoglobin trend over the first three days for each dose level revealed a strong linear relationship. (r=0.98, p<0.0001)

FIG. 4: The final 4-week relative vessel density demonstrated a strong inverse relationship with initial three day deoxyhemoglobin slope for each radiation dose. (r=0.91, p<0.0001)

FIG. 5: Maximal skin reaction score by day 14 was strongly correlated to the initial three day deoxyhemoglobin slope for each radiation dose. (r=0.98, p<0.0001)

FIG. 6: Macroscopic, histological, and vsHSI comparisons at three days post burn. Left to right columns are unburned skin, intermediate dermal burn, deep dermal burn, and full-thickness burn. Rows top to bottom are gross view, vsHSI (OxyHb parameter), histology 10× magnification and stained with Masson's trichrome.

FIG. 7: Total Hemoglobin (tHb) Variations in Three Depths of Burn. tHb is relative to its pre-burn baseline for the three depths of burn, measured over the first 72 hours post injury. All values are expressed as mean±standard deviation of the mean. Statistical significance of p<0.05 between ID and DD burns is indicated with an (*) asterisk.

FIG. 8: Deoxygenated Hemoglobin (DeOxyHb) Variations in Three Depths of Burn. DeOxyHb is relative to its pre-burn baseline for the three depths of burn, measured over the first 72 hours post injury. All values are expressed as mean±standard deviation of the mean. Statistical significance of p<0.05 between ID and DD burns is indicated with an (*) asterisk.

FIG. 9: Oxygenated Hemoglobin (OxyHb) Variations in Three Depths of Burn. OxyHb is relative to its pre-burn baseline for the three depths of burn, measured over the first 72 hours post injury. All values are expressed as mean±standard deviation of the mean. Statistical significance of p<0.05 between ID and DD burns is indicated with an (*) asterisk.

FIG. 10: Oxygen Saturation (SatO2) Variations in Three Depths of Burn. SatO2 is relative to its pre-burn baseline for the three depths of burn, measured over the first 72 hours post injury. All values are expressed as mean±standard deviation of the mean. Statistical significance of p<0.05 between ID and DD burns is indicated with an (*) asterisk.

FIG. 11: Oxygen Saturation (SatO2) compared to final burn depth. SatO2 at 1 hour plotted against final burn depth 72 hours post injury demonstrates a non-linear correlation (R²=0.99975, p<0.001).

### DETAILED DESCRIPTION OF THE INVENTION

The invention provides hyperspectral imaging-based methods that enable effective, efficient and non-invasive detection, characterization and prediction of the effect of thermal and ionizing radiation exposure in tissue. By complete visualization and quantification of oxygenation and perfusion changes in thermal burn or ionizing radiation impacted skin, the method of the invention enables rapid identification of individuals exposed to such exposures and allows early prediction of extent of injury in normal tissue after exposure.

Thermal injury is divided into four degrees of depth or severity: superficial, superficial-partial, deep-partial, and full thickness burns. First-degree burns, also called superficial burns, only involve the uppermost layer of skin, the epidermis. These burns heal within days and do not result in scarring. A blistering sunburn is an example of a superficial burn. Second-degree burns involve the entire epidermis and part of the dermis. Depending on the extent of dermal involvement, these burns may be further divided into superficial and deep partial thickness burns. While superficial-partial burns generally heal without surgical intervention, deep-partial thickness burns may resist healing and surgical intervention may be required. Third degree, or full-thickness, burns require surgical excision and subsequent skin grafting.

A promising parameter for burn depth assessment is its correlation with skin perfusion: superficial burn injury causes an inflammatory response and thus increased perfusion to the burn site, while deeper burns are prone to destroy the dermal microvasculature and thus a decrease in perfusion follows. Furthermore, the distinction between destroyed versus preserved blood supply has impact over the future viability and recovery of tissue. However, despite advances in the field of burn diagnostics, a reliable non-invasive method to aid in early burn depth diagnosis still does not exist. (Alsbjörn, et al. 1984 Scandinavian Journal of Plastic and Reconstructive Surgery and Hand Surgery 18.1: 75-79; Niazi, et al. 1993 Burns 19.6: 485-489; Pape, et al. 2001 Burns 27.3: 233-239; Droog, et al. 2001 Burns 27.6: 561-568; Park, et al. Burns (2013) 655-661; Monstrey, et al. 2008 Burns 34.6: 761-769; Qin, et al. 2012 Biomedical Optics Express 3.3: 455-466; Sowa, et al. 2001 Burns 27.3: 241-249; Cross, et al. 2007 Wound Repair and Regeneration 15.3: 332-340.)

Exposure to ionizing radiation can also have profound biological consequences to the skin. Scenarios for radiation exposure range from external beam radiotherapy for oncologic treatment to uncontrolled release events such as a nuclear attack or accident. The disasters it the Fukushima and Chernobyl emphasize the real possibility of the latter scenario. Cutaneous skin reactions in response to ionizing radiation exposure are major consequences in both cases. These skin reactions in the acute phase can range from mild skin erythema to ulceration. For radiotherapy alone, it has been reported that a majority of patients receiving radiotherapy experience some degree of skin reaction. (Murray, et al. 2011 Radiother Oncol. 99.2: 231-4.)

The precise etiology of ionizing radiation-induced skin injury remains unclear. Multiple theories proposed include direct cellular injury, cell signaling dysregulation, and ischemia. Recently, the literature has suggested that cutaneous ischemia may be the predominant characteristic that links both acute and chronic-phase injuries. However, these studies have all been limited as they use only a single large dose of ionizing radiation. (Marx, et al. 1990 Am J Surg. 160.5:519-24; Martin, et al. 2000 Int J Radiat Oncol Biol Phys. 47.2: 277-90; Aitasalo, et al. 1986 Plast Reconstr Surg. 77.2: 256-67; Thanik, et al. 2011 Plast Reconstr Surg. 127.2: 560-8; Doll, et al. 1999 Radiother Oncol. 51.1: 67-70.)

Recently, a novel method for assessing radiation-induced skin injury has been reported. (Chin, et al. 2012 Journal of Biomedical Optics. 17.2: 026010; Chin, et al. 2013 Plast Reconstr Surg. 131.4: 707-16; WO 2013/082192A1 by Chin) For example, using a hairless mouse and a single fixed-dose of surface beta-irradiation, a reliable model of radiation-induced skin injury has been generated. Using hyperspectral imaging technology, characteristic changes have been identified in cutaneous perfusion that are reproducible in magnitude and in timing, preceding the visible appearance of skin injury.

Two significant issues remain: (1) the relationship between the dose of cutaneous irradiation and the magnitude of cutaneous perfusion changes observed over time and (2) the relationship between the magnitude of early cutaneous perfusion changes observed over time and the development of acute skin injury post-irradiation. The latter posses a significant clinical problem since the development of acute skin reactions during radiotherapy usually signals a treatment break. It has been estimated that a treatment break of more than a week during breast cancer radiotherapy can have significant negative impact on recurrence rate and overall survival. (Bese, et al. 2007 Journal of BUON 12.3: 353-9; Bese, et al. 2005 Oncology. 69.3: 214-23.) Therefore, the ability to detect and predict high-risk areas for developing skin reactions and change the treatment plan may prevent the need for these detrimental treatment breaks.

Current standard of practice for evaluating burn injury involve visual estimation by clinical exam. Previous groups have demonstrated the ability of hyperspectral imaging to assess the current perfusion and oxygenation patterns of burned skin, but no predictive model for maximal burn depth was generated from this work. Hyperspectral imaging and other non-invasive techniques such as laser Doppler flowmetry have been used to demonstrate early changes in perfusion and oxygenation but no methods have been developed to use this information to predict a precise level of maximal burn depth which forms several days later. In addition, there are no existing methods for prediction of skin reactions (acute or chronic) after exposure to ionizing radiation.

A unique aspect of this invention is the application of an existing technology, hyperspectral imaging, to early prediction of injury in normal tissue after exposure to thermal injury or ionizing radiation. These normal tissues include skin and any external surface of the body, e.g., eyes, nails, hair, etc. For thermal injury, maximum level of burn injury is defined as the percentage of dermis presenting with burn injury 72 hours after thermal exposure. The invention described herein utilizes hyperspectral imaging for the prediction of maximum level of burn injury in normal tissue as well as the prediction of acute and chronic skin reactions secondary to radiation exposure.

In particular, the invention employs changes in the spectral signature of skin, including those representative of oxy-hemoglobin and deoxy-hemoglobin levels, to assess thermal exposure and predict the depth of burn injury. The spectral signature is represented by selected specific wavelengths, for example, of visible and infrared light (e.g., 350 nm-1200 nm). This invention allows us to utilize these detected changes to determine what tissue has been exposed to thermal burn and predict the clinical presentation of maximal depth of burn or ionizing radiation injury. Using the method disclosed herein, one can reliably predict the maximal depth of injury as early as 1 hour after thermal exposure, a task not possible with existing methods.

For radiation injury, acute skin injury is defined as an erythema, moist or dry desquamation, or ulceration that occurs days to weeks after radiation exposure. Chronic injury refers to fibrosis, decreased vasculature, and chronic ulceration that forms months to years later. The invention utilizes changes in the spectral signature of skin, including those representative of oxy-hemoglobin and deoxy-hemoglobin levels, to assess radiation exposure and predict acute and chronic skin reactions. The spectral signature is represented by selected specific wavelengths, for example, of visible and infrared light (e.g., 350 nm-1200 nm). This invention allows us to utilize these detected changes to determine what tissue has been exposed to predict the clinical presentation of acute or chronic skin injury. Using the method disclosed herein, one can reliably predict the maximal depth of injury as early as 72 hours hour after radiation, a task not possible with existing methods.

The novel method of the invention relies on changes in the oxy- and deoxy-hemoglobin levels as assessed by their reflectance and absorbance of visible light in areas of thermal and radiation exposure to predict subsequent injury. The disclosed method is rapid and non-invasive and the results are available at the point-of-care and allow for immediate triage and decision-making ability. With regards to the ability of hyperspectral imaging to characterize changes in perfusion and oxygenation in normal tissue, the invention allows for the simultaneous assessment of oxygenation and perfusion changes.

For burn patients and healthcare providers, the invention offers significant benefits including the early and accurate prediction of maximal level burn injury, for instance, in emergency department and trauma settings. Early assessment of maximal burn depths enables more accurate assessment of total body surface area burned and therefore guides fluid resuscitation. In addition, early prediction of thermal burns allows for close monitoring and earlier excision of full thickness burns thereby minimizing chances for complications such as major infection or septic shock. Early prediction of high-risk burn areas may also identify areas that might respond to mitigating therapeutics.

Major benefits of the disclosed method for skin reactions secondary to ionizing radiation include providing radiation oncologists with an indication for areas that are high risk for developing a skin reaction during the treatment course, which enable the oncologist to refine the treatment plan to avoid a skin reaction without a detrimental treatment break. Additionally, the method produces prediction of areas that may be prone to chronic ulceration or infection years later after exposure to ionizing radiation. This is particularly important to plastic surgeons who may be consulted to provide wound care treatment or reconstructive procedures to the affected area.

In one aspect, the invention generally relates to a method for predicting a maximal depth of thermal burn injury formation in superficial tissue of a subject. The method includes: acquiring photographic imagery of one or more areas of superficial tissue of the subject at one or more wavelengths of light and one or more time points; and characterizing the obtained photographic imagery to measure one or more physiological properties in the one or more areas of superficial tissue to predict the maximal depth of burn injury formation in superficial tissue of the subject.

In certain embodiments, the one or more physiological properties are selected from tissue oxygenation and perfusion levels after exposure to thermal injury.

The one or more wavelengths of light may be any suitable wavelength, for example, selected from the range from about 350 nm to about 1,200 nm (e.g., from about 350 nm to about 900 nm, from about 350 nm to about 700 nm, from about 400 nm to about 1,200 nm, from about 550 nm to about 1,200 nm).

The photographic imagery may be obtained at a time suitable for the application at hand. In certain embodiments, the photographic imagery is obtained within a time frame from about 1 hour to about 48 hours (e.g., from about 1 hour to about 36 hours, from about 1 hour to about 24 hours, from about 1 hour to about 24 hours, from about 1 hour to about 12 hours, from about 1 hour to about 6 hours, from about 1 hour to about 3 hours, from about 6 hours to about 48 hours, from about 6 hours to about 36 hours, from about 6 hours to about 24 hours, from about 6 hours to about 12 hours) after a thermal exposure to predict the maximum burn depth. In certain embodiments, the photographic imagery is obtained within a time frame from about 6 hours to 1 day after a thermal exposure. In certain embodiments, the photographic imagery is obtained after 1 day after a thermal exposure.

In certain embodiments, measuring one or more physiological properties includes detecting and quantifying the level of oxygenated hemoglobin and an increase or decrease in measured levels of oxygenated hemoglobin in the burned area of the subject is used as a biomarker to predict maximum burn depth.

In certain embodiments, measuring one or more physiological properties includes detecting and quantifying the level of de-oxygenated hemoglobin and an increase or decrease in measured levels of de-oxygenated hemoglobin in burned area of a subject is used as a biomarker to predict maximum burn depth.

In certain embodiments, measuring one or more physiological properties comprises detecting and quantifying the level of tissue oxygen saturation and an increase or decrease in measured levels of tissue oxygen saturation in the burned area of a subject is used as a biomarker to indicate predict maximum burn depth.

In certain embodiments, measuring one or more physiological properties comprises detecting and quantifying the level of total hemoglobin and an increase or decrease in measured levels of total hemoglobin in the burned area of a subject is used as a biomarker to predict maximum burn depth.

Characterization of the obtained photographic imagery may be performed in conjunction with assessment of collagen, lipids, water, or another naturally occurring molecules.

In certain embodiments, the method further includes characterizing the obtained photographic imagery to measure one or more physiological properties in the one or more areas of superficial tissue to estimate total body surface area of a patient's burned skin.

In another aspect, the invention generally relates to a biomedical imaging method for predicting acute skin reactions after exposure to ionizing radiation. The method includes: acquiring photographic imagery of one or more areas of superficial tissue of the subject at one or more wavelengths of light and one or more time points; and characterizing the obtained photographic imagery to detect changes in tissue oxygenation and perfusion levels of the subject to predict acute skin reactions.

Various acute skin reactions can be detected and evaluated by the method of the invention, for example, erythema, moist or dry desquamation, or ulceration.

The one or more wavelengths of light may be any suitable wavelength, for example, selected from the range from about 350 nm to about 1,200 nm (e.g., from about 350 nm to about 900 nm, from about 350 nm to about 700 nm, from about 400 nm to about 1,200 nm, from about 550 nm to about 1,200 nm).

The photographic imagery may be obtained at a time suitable for the application at hand. In certain embodiments, the photographic imagery is obtained within a time frame from about 3 to about 5 days (e.g., about 3 days, about 4 days, about 5 days) after exposure to ionizing radiation to predict acute skin reaction occurring within one month.

In certain embodiments, measuring one or more physiological properties comprises detecting and quantifying oxygenated hemoglobin levels and an increase or decrease in measured levels of oxygenated hemoglobin in the exposed area of a subject is used as a biomarker to predict acute skin reaction.

In certain embodiments, measuring one or more physiological properties comprises detecting and quantifying de-oxygenated hemoglobin levels and an increase or decrease in measured levels of de-oxygenated hemoglobin in the exposed area of a subject is used as a biomarker to predict acute skin reaction.

In certain embodiments, measuring one or more physiological properties comprises detecting and quantifying the level of tissue oxygen saturation and an increase or decrease in measured levels of tissue oxygen saturation in the burned area of a subject is used as a biomarker to indicate predict acute skin reaction.

In certain embodiments, measuring one or more physiological properties comprises detecting and quantifying the level of total hemoglobin and an increase or decrease in measured levels of total hemoglobin in the burned area of a subject is used as a biomarker to predict acute skin reaction.

Characterization of the obtained photographic imagery may be performed in conjunction with assessment of collagen, lipids, water, or another naturally occurring molecules.

In yet another aspect, the invention generally relates to a biomedical imaging method for predicting acute skin reactions after exposure to thermal injury. The method includes: acquiring photographic imagery of one or more areas of superficial tissue of the subject at one or more wavelengths of light and one or more time points; and characterizing the obtained photographic imagery to detect changes in tissue oxygenation and perfusion levels of the subject to predict acute skin reactions.

Various acute skin reactions can be detected and evaluated by the method of the invention, for example, erythema, moist or dry desquamation, or ulceration.

The one or more wavelengths of light may be any suitable wavelength, for example, selected from the range from about 350 nm to about 1,200 nm (e.g., from about 350 nm to about 900 nm, from about 350 nm to about 700 nm, from about 400 nm to about 1,200 nm, from about 550 nm to about 1,200 nm).

The photographic imagery may be obtained at a time suitable for the application at hand. In certain embodiments, the photographic imagery is obtained within a time frame from about 1 hour to about 48 hours (e.g., from about 1 hour to about 36 hours, from about 1 hour to about 24 hours, from about 1 hour to about 24 hours, from about 1 hour to about 12 hours, from about 1 hour to about 6 hours, from about 1 hour to about 3 hours, from about 6 hours to about 48 hours, from about 6 hours to about 36 hours, from about 6 hours to about 24 hours, from about 6 hours to about 12 hours) after a thermal exposure to predict the maximum burn depth. In certain embodiments, the photographic imagery is obtained within a time frame from about 6 hours to 1 day after a thermal exposure. In certain embodiments, the photographic imagery is obtained after 1 day after a thermal exposure.

In certain embodiments, measuring one or more physiological properties includes detecting and quantifying the level of oxygenated hemoglobin and an increase or decrease in measured levels of oxygenated hemoglobin in the burned area of the subject is used as a biomarker to predict maximum burn depth.

In certain embodiments, measuring one or more physiological properties includes detecting and quantifying the level of de-oxygenated hemoglobin and an increase or decrease in measured levels of de-oxygenated hemoglobin in burned area of a subject is used as a biomarker to predict maximum burn depth.

In certain embodiments, measuring one or more physiological properties comprises detecting and quantifying the level of tissue oxygen saturation and an increase or decrease in measured levels of tissue oxygen saturation in the burned area of a subject is used as a biomarker to indicate predict maximum burn depth.

In certain embodiments, measuring one or more physiological properties comprises detecting and quantifying the level of total hemoglobin and an increase or decrease in measured levels of total hemoglobin in the burned area of a subject is used as a biomarker to predict maximum burn depth.

Characterization of the obtained photographic imagery may be performed in conjunction with assessment of collagen, lipids, water, or another naturally occurring molecules.

The subject may be any suitable species, including a human and a non-human animal.

A computer and algorithm may be included in the system for image processing and data analysis, particularly in characterizing the obtained photographic imagery.

In certain embodiments, the method further includes determining course of medical treatment or segregation of individual subjects into groups for triage in a mass casualty scenario. In certain embodiments, the method further includes segregating individual subjects into groups for triage in a mass casualty scenario.

### EXAMPLES

### Example 1

### Hyperspectral Imaging as an Early Biomarker for Radiation Exposure and Microcirculatory Damage

The study demonstrates that early measurement of cutaneous deoxygenated hemoglobin levels after radiation exposure is a useful biomarker for dose reconstruction and also for chronic microvascular injury. Changes in deoxygenated hemoglobin can also be correlated to acute skin reactions before any are visible.

### Animals and Irradiation Procedure

All handling of and procedures performed with animals was done in accordance with a protocol (UMass IACUC Protocol #A2354) approved by our Institutional Animal Care and Use Committee. Prior to this experiment, a model of acute radiation-induced skin injury was established. (Chin, et al. 2012 J. Biomedical Optics 17(2):026010.) The radiation source utilized was a Strontium-90 beta-emitter with an active diameter of 9 mm, which created an 8 mm diameter skin injury. This beta-emitter source delivers less than 10% of the total dose deeper than 3.6 mm into the skin, avoiding injury to internal organs.

Hairless, immunocompetent adult mice (SKH-1 Elite, Charles River Laboratories, Wilmington, Mass.) were used. Mice (n=66) were equally divided into 6 groups. Mice received one of six pre-specified doses of ionizing radiation respectively: 0, 5, 10 , 20, 35, and 50 Gy.

On day 0 mice were anesthetized. Tattooing was performed bilaterally on the dorsal flank skin of the mice to act as fiducial marks. Cutaneous perfusion was assessed using HSI prior to irradiation as described below. This pre-irradiation assessment served as a baseline level for subsequent comparison. Mice then received a single pre-specified dose of irradiation as described above. Accurate dose-delivery was ensured prior to animal irradiation using radiochromic film dosimetry. Following irradiation, mice were recovered and housed individually.

### Hyperspectral Imaging for Evaluation of Cutaneous Perfusion

Evaluation of cutaneous perfusion was accomplished using a novel application of an existing technology, HSI. HSI is a method of wide-field diffuse reflectance spectroscopy that utilizes a spectral separator to vary the wavelength of light entering a digital camera and provides a diffuse reflectance spectrum for every pixel. These spectra are then compared to standard transmission solutions to calculate the concentration of deoxy-hemoglobin (DeoxyHb) in each pixel, from which spatial maps of these parameters are constructed.

Using HSI, cutaneous perfusion was analyzed over the first three days after radiation exposure. Skin reactions were then evaluated twice weekly for the first 14 days and then weekly through 28 days post-irradiation. At the time of each evaluation, mice were anesthetized and maintained at standard body temperature. The OxyVu-2 device (HyperMed, Greenwich, Conn.) was utilized for HSI acquisitions. The OxyVu-2-generated spatial maps of tissue DeoxyHb were used for quantification of cutaneous perfusion. These maps were analyzed with MATLAB R2010b (Mathworks Inc., Natick, Mass.). Mean values of DeoxyHb were calculated for a 79-pixel area corresponding to the irradiated area on each flank. This 79-pixel area was determined precisely over time with reference to the fiducial tattoo marks that were placed prior to irradiation.

Similarly, areas of non-irradiated contralateral flank skin were quantified to ensure that any changes in perfusion observed in the irradiated skin were not due to natural variations or systemic phenomena.

Values for DeoxyHb parameters for post-irradiation time points are expressed as relative to pre-irradiation values within the same area of skin. Mean relative values reported hereafter reflect the average of relative levels for all subjects within a dose group at a specified timepoint.

### Post-Irradiation Tissue Analysis

On day 28 post-irradiation, mice were euthanized following cutaneous perfusion assessment. Immediately post-mortem, irradiated and non-irradiated skin from both flanks was harvested. Tissue was fixed en bloc in 10% neutral-buffered formalin solution and kept at 4° C. overnight for paraffin embedment. Paraffin-embedded sections were re-hydrated though a decreasing alcohol series and stained for vasculature as described previously. (Chin, et al. 2013 Plast Reconstr Surg. 131(4):707-16.) Primary antibody (PECAM-1) for vasculature staining (BD Pharmingen, San Jose, Calif.) was incubated at 4° C. overnight. Signals were intensified by using a tyramide amplification system (PerkinElmer, Boston, Mass.) and activated with DAB Chromogen (Dako North America Inc., Carpinteria, Calif.). Slides were counterstained with hematoxylin.

Digital images were obtained from the center of all stained skin sections at 10× magnification with an Olympus BX53 microscope (Olympus America Inc., Center Valley, Pa.). Two blinded reviewers quantified vessel density using 3 random standard-sized fields and results were averaged. Vessel densities for each animal were expressed as a ratio between the irradiated sample and its respective contralateral non-irradiated internal control, yielding normalized mean vessel densities.

### Statistical Analysis

For hyperspectral data, plots of perfusion data are expressed as the relative mean unless otherwise specified. Changes in DeoxyHb and dose were correlated with cutaneous blood vessel densities using linear regression models. Statistical significance was assumed for p values less than 0.05.

### Macroscopic Skin Reaction

Mice were irradiated as previously described without complication. Mice gained weight appropriately following irradiation and no morbidity or mortality was observed. Skin reactions began forming in all groups by approximately one week post-irradiation. Maximal skin reactions were observed by day 14 in all groups and scored using the Radiation Therapy Oncology Group toxicity scoring system, which describes skin reactions from erythema to ulceration over a four-point scale. (Salvo, et al. 2010 Current oncology 17(4):94-112.) Maximal skin reactions were observed to be characterized by erythema in the 5 and 10 Gy groups. Dry desquamation was observed as the maximal skin reaction of mice receiving 20 and 35 Gy. In the 50 Gy group, moist desquamation was observed in all irradiated areas.

### Changes in Microvascular Density

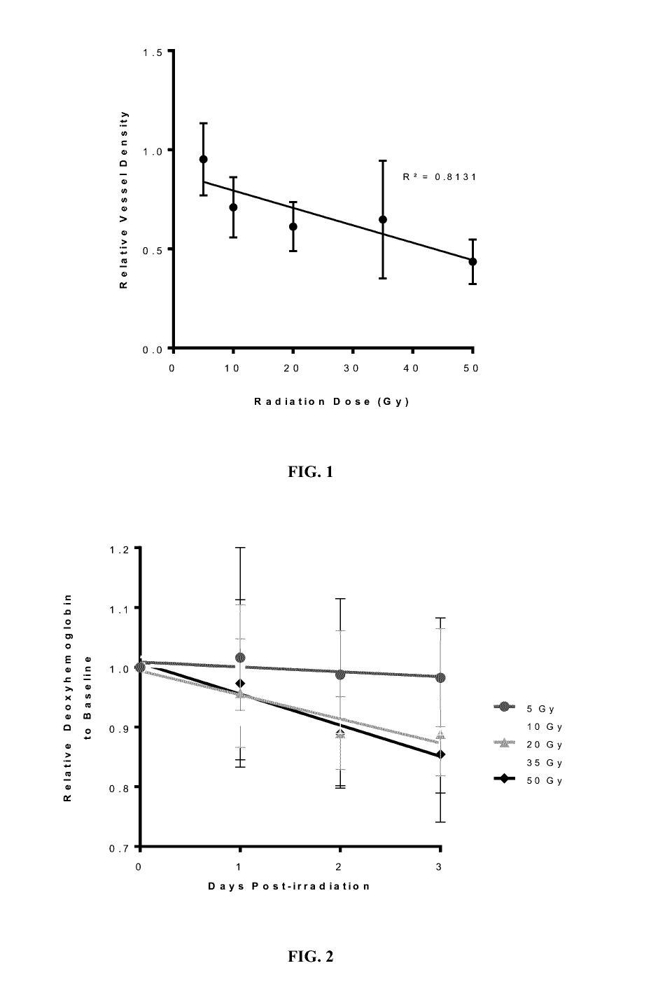

A pronounced reduction in cutaneous vascular densities at four weeks following irradiation was observed. CD31 immunohistochemistry demonstrated an inverse correlation (r=0.90, p<0.0001) between dose and vessel reduction severity, with vessel counts decreasing as the dose increased. (FIG. 1)

### Relationship of Early Deoxygenated Hemoglobin Changes and Radiation Dose

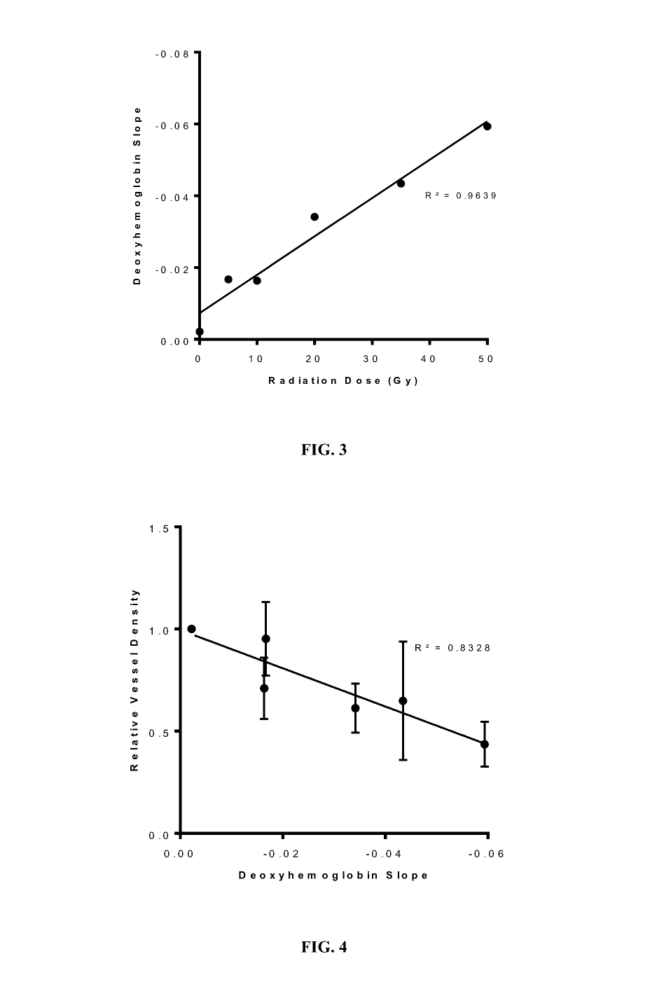

Using HSI analysis, early changes in deoxygenated hemoglobin levels were observed during the first three days post-irradiation in all groups. An acute decrease in deoxygenated hemoglobin was seen for each dose over the first 3 days before any visible skin reactions were observed. (FIG. 2) When further examining the behavior of deoxygenated hemoglobin over the first 3 days, it was noted that the slope changed linearly with increasing radiation dose exposure (r=0.98, p<0.0001). (FIG. 3)

Correlation between Early Deoxygenated Hemoglobin Changes and Microvascular Density

Using the relationships between early DeoxyHb change with dose, it was also observed that deoxygenated hemoglobin decrease at 3 days had a strong linear association with microvascular density by 4 weeks for the given doses, indicating that early deoxygenated hemoglobin response could be predictive of final degree of microvascular damage. There was a highly significant correlation (r=0.91, p<0.0001) between these early changes in deoxygenated hemoglobin and late vascular injury severity assessed. (FIG. 4)

### Relationship Between Early Deoxygenated Hemoglobin Changes and Skin Reaction

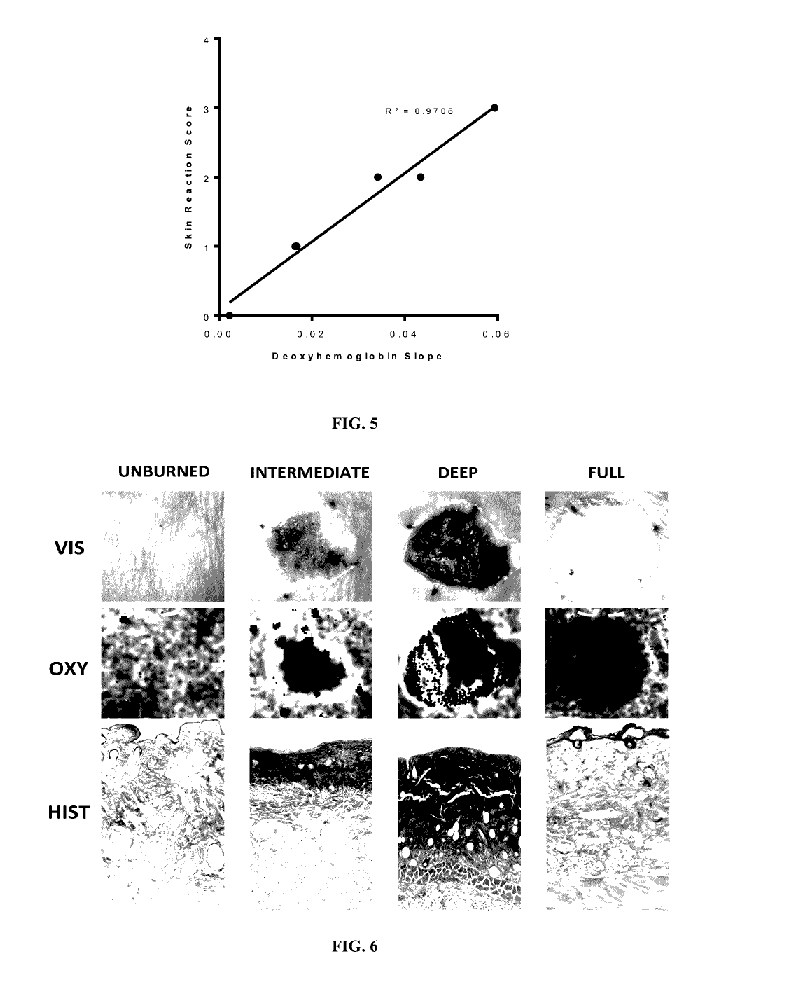

In addition, when examining the relationship between deoxygenated slope change in the first 3 days and maximal skin reaction at 14 days, there was a strong linear correlation (r=0.98, p<0.0001). (FIG. 5) This suggests that early decreases seen during serial assessment of deoxygenated hemoglobin may be related to the degree of maximal skin reaction after radiation exposure.

The current study demonstrated that over a large exposure gradient, the initial radiation dose appears to be directly correlated to the degree of microvascular injury. This finding is consistent with results by Haubener et al who showed that varying doses of single fraction radiation exposure had an increasing effect on diminishing endothelial cell number in vitro.

Furthermore, it was found that a strong relationship exists between deoxygenated hemoglobin response and final vessel density after radiation exposure. This suggests the possibility of using early monitoring of deoxygenated hemoglobin for prediction of chronic vascular damage.

In parallel, these results also indicate that deoxygenated hemoglobin response may be related to formation of acute skin reaction seen weeks after initial radiation exposure. No previous studies have been able to identify a biomarker that predicts the formation of an adverse skin reaction after exposure to radiation. If supported by clinical studies, applications of this finding could result in improved skin monitoring of patients receiving both therapeutic and diagnostic radiologic procedures. Currently, the development of acute skin reactions during radiotherapy usually signals a treatment break. It has been estimated that a treatment break of a more than a week during breast cancer radiotherapy can negatively impact recurrence rate and overall survival. Therefore, the ability to detect and predict high-risk areas or high-risk patients for developing skin reactions earlier and change the treatment plan accordingly, may prevent the need for these detrimental treatment breaks.

### Example 2

### Hyperspectral Imaging as an Early Prediction of Maximal Burn Depth After Thermal Injury

This example was to characterize dermal perfusion and oxygenation in three sequential depths of burn over a dynamic, three-day period after burn injury, and to assess whether vsHSI could differentiate depths of injury, based on any of the parameters it quantifies: oxyHb, deoxyHb, tHb, or StO₂.

### Animals and Thermal Burn Procedure

All handling of and procedures performed with animals was done in accordance with a protocol (UMass IACUC Protocol #A2454) approved by our Institutional Animal Care and Use Committee.

One hundred and seven hairless immune-competent, adult male mice (SKH-1 Elite, Charles River Laboratories, Wilmington, Mass.) were used. Anesthesia for thermal burn procedure and imaging was performed with a mixture of ketamine (55 mg/kg) and xylazine (5 mg/kg). Tattoo marks were placed on the dorsum of the mice to act as fiducial marks. After anesthesia, the dorsal skin was distracted from the body, and a 1.5 cm diameter brass rod of 82 grams heated to 50 (n=12), 55 (n=12), 60 (n=26), 70 (n=23), 80 (n=12), or 90° C. (n=22) was applied to the dorsum of the anesthetized mice (gravity only) for 10 seconds creating thermal injury of graded severity. Animals received subcutaneous, slow release buprenorphine (0.2 mg/kg) and intraperitoneal 1 ml of normal saline after the burn procedure. Twelve mice randomly selected from each burn group were sacrificed at 72 hours after burn injury (n=72) and biopsy samples were taken for histological analysis. The rest were sacrificed at a later timepoint out of the scope of this paper.

### Histological Burn Depth Analysis

Punch skin biopsies were taken from the center of burns at 72 hours post injury to evaluate burn progression over the first 3 days post injury. (Jackson 1953 The British Journal of Surgery 40(164):588-596; Tobalem, et al. 2013 Journal of Plastic, Reconstructive & Aesthetic Surgery 66(2):260-266.) Biopsies fixed in buffered formalin, embedded in paraffin and 10 micron sections stained with Masson's Trichrome, a stain favorable in partial burns due to its magenta-red coloring of denatured collagen, indicative of the extent of the burn. (Chvapil, et al. 1984 Plastic and Reconstructive Surgery 73(3):438-441.) Images were taken with an Olympus microscope at 10× magnification. Burn depth was measured with a linear tool of Image J from every picture at three places in each image: left, center, and right. Percent burn depth was defined as depth of denatured collagen divided by depth to Panniculus Carnosus muscle×100. Averages were taken of all the depths measured for each temperature of burn.

Evaluation of Cutaneous Perfusion and Oxygenation With Hyperspectral Imaging in the Visible Spectrum (vsHSI)

Perfusion and oxygenation parameters of the injured skin were measured with vsHSI, a non-invasive method of wide-field, diffuse reflectance spectroscopy at baseline before the burn, and at 2 minutes, 1 hour, 24 hours, 48 hours, and 72 hours after burn injury.

The OxyVu2™ device (HyperMed, Greenwich, Conn.) utilizes a spectral separator to vary the wavelength of visible light entering a digital camera and provides a diffuse reflectance spectrum for every pixel. These spectra are then compared to standard transmission solutions to calculate the concentration of oxygenated hemoglobin (oxyHb) and deoxygenated hemoglobin (deoxyHb) in each pixel, from which spatial maps of these parameters are constructed and used for quantification of cutaneous perfusion and oxygenation. These maps were analyzed with MATLAB R2010b (Mathworks Inc., Natick, Mass.). Mean values of oxyHb and deoxyHb were calculated for a 79-pixel area corresponding to the burned skin. This 79-pixel area was determined precisely over time with reference to the fiducial tattoo marks that were placed prior to burn injury. OxyHb and deoxyHb values were summed to yield total tissue hemoglobin (tHb), which represents total blood volume and thus total perfusion in the area of skin examined. Tissue oxygen saturation (StO₂) was calculated as oxyHb divided by tHb.

Post-burn oxyHb, deoxyHb, tHb, and StO₂ values are expressed as relative to pre-burn values within the same area of skin to account for oxygenation differences between animals at baseline.

### Statistical Analysis

Descriptive statistics were conducted on all of the measures of hemoglobin concentration: oxyHb, deoxyHb, tHb and StO₂ at the burn site at each time point, including 2 minutes, 1 hour, 24 hours, 48 hours and 72 hours following the induced burn. A series of Kruskal Wallis tests were used to determine whether there are any significant differences in hemoglobin concentration measures by the class of burn. A separate test for each measure of hemoglobin concentration at each separate time measure was performed. Non-parametric post-hoc Wilcoxon rank sum tests comparing whether a hemoglobin measure was different between two specific classes of burn at the same time point was applied. Post-hoc tests were only conducted when the omnibus Kruskallis Wallis test found a significant difference in hemoglobin measures by the 3 classes of burn: intermediate dermal, deep dermal, and full-thickness. All statistical tests were performed using Stata 13 (StataCorp. 2013. Stata Statistical Software: Release 13. College Station, Tex. StataCorp LP).

### Histological Assessment

Three discrete levels of burn depth were identified histologically. Fifty-degree and fifty-five degree Celsius groups produced no injury. The sixty-degree group resulted in 58% (±18%) of dermal damage that was classified as intermediate dermal (ID) burn. Seventy-degree group resulted in 80% (±14%) of dermal damage, which was classified as deep dermal (DD) burn. Both eighty and ninety-degree groups resulted in full-thickness (FT) injury 95% (±13%) and 99% (±2%), respectively (FIG. 6).

Evaluation of Cutaneous Perfusion and Oxygenation With vsHSI

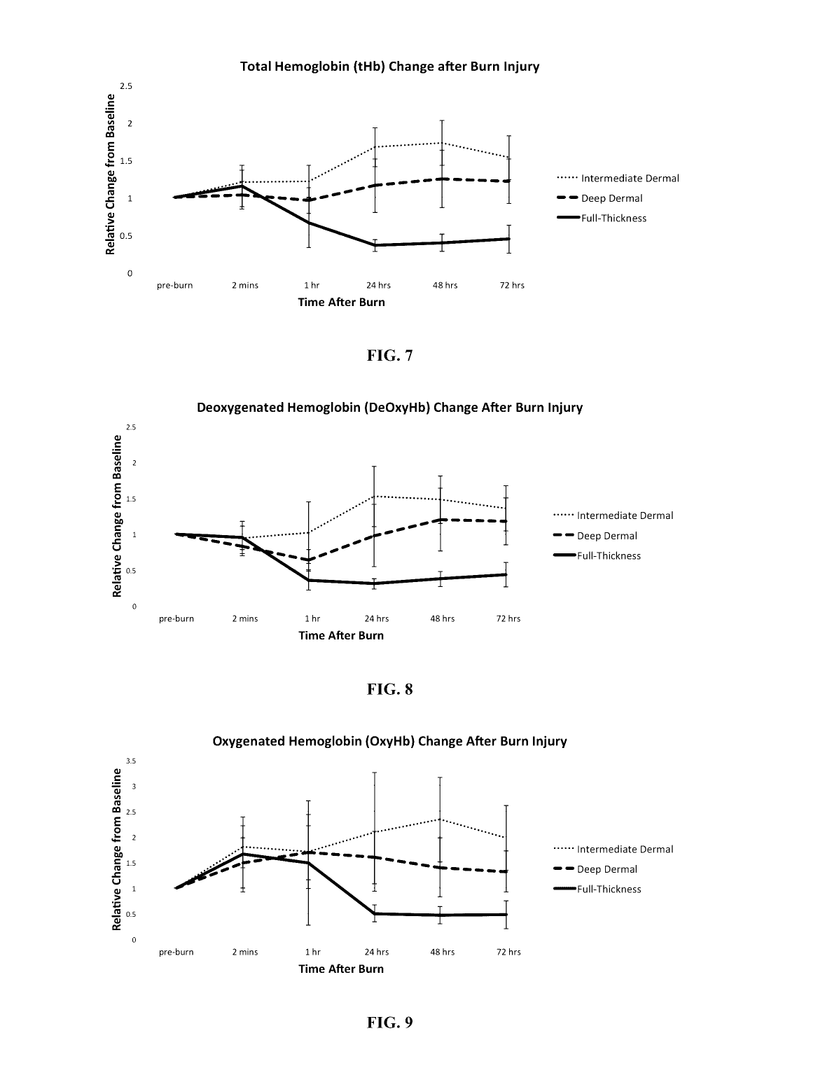

Total Hemoglobin (tHb). The ID burn group exhibited a rise in tHb beginning at 2 minutes post injury, peaked at 48 hrs with a 1.73 fold increase over baseline, and continued to be elevated until the end of the experiment at 72 hours with a 1.53 fold increase over baseline. The DD group also exhibited an increase in tHb over baseline levels, though to a lesser extent than the ID group, with a tHb peak at 48 hours of 1.25 fold increase over baseline levels and a 1.22 increase at 72 hours. FT injury had a fall in tHb beginning at 1 hour with tHb being 0.67 of baseline levels and reached the lowest point at 24 hours with 0.36 of baseline levels; at 72 hours tHb levels were 0.44 of original levels (FIG. 7). tHb was statistically different (p<0.05) between ID and DD as well as DD and FT at all time points; it was also significant between ID and FT at all timepoints except at the 2 minute mark.

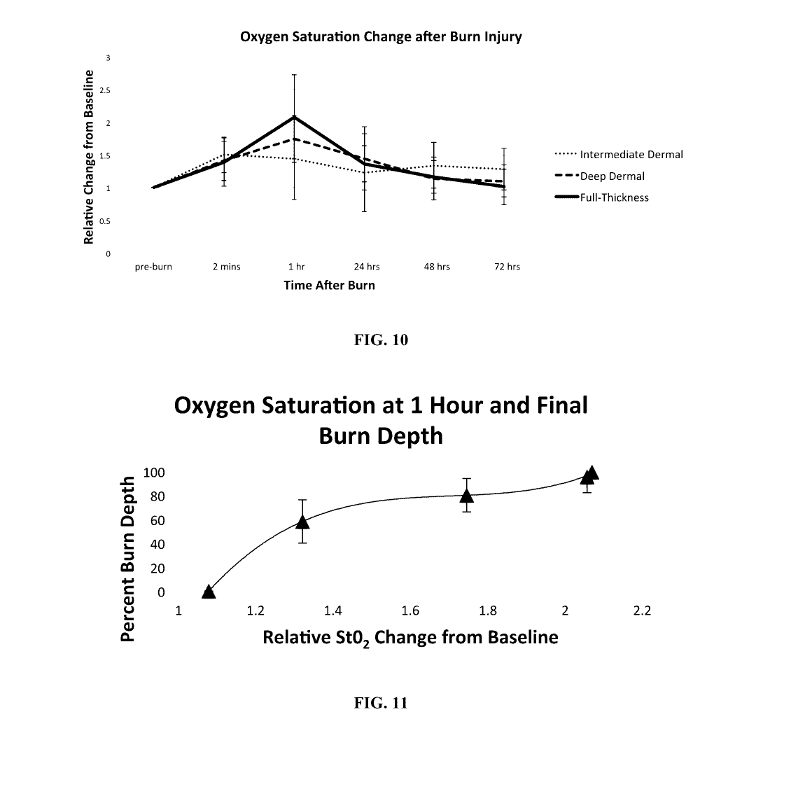

Oxygenated and Deoxygenated Hemoglobin. FIG. 9 shows the changes in deoxygenated hemoglobin (deoxyHb) and FIG. 10, changes in oxygenated hemoglobin (oxyHb), of each burn depth, over 72 hours post injury. The ID burn group had the greatest rise in both oxyHb and deoxyHb. ID group's oxyHb peaked at 48 hours at 1.34 fold increase over baseline, and its deoxyHb peaked at 24 hours with 1.52 increase. DD burn group had a peak of oxyHb at 1 hour at 1.69 over baseline, and then it trended down, with 1.32 over original levels at 72 hours. DD's deoxyHb initially decreased after injury, with 0.63 of baseline levels, at 1 hour. Then it increased to pre-burn levels by 24 hours, and to 1.20 above baseline levels at 48 and 72 hours. FT burn group had an increase in oxyHb of 1.48 at 1 hour, followed by a decrease with 0.48-0.50 of baseline levels at 24, 48, and 72 hours. The deoxyHb in the FT group decreased at 1 hour to 0.36 and at 24 hours to 0.31 of baseline levels; at 48 and 72 hours deoxyHb recovered slightly to 0.38 and 0.44 of baseline levels, respectively (FIG. 8 and FIG. 9). DeoxyHb was statistically different (p<0.05) between ID and DD at all but the 2 minute and 72 hour time points, and between DD and FT as well as ID and FT, at all but the 2 minute time point. OxyHb was significant between the ID and DD groups at 48 and 72 hours, and between the DD and FT as well as ID and FT, at 24, 48, and 72 hours.

Oxygen Saturation. FIG. 11 depicts saturation changes in the three burn-depth groups post injury. At 1-hour post injury, FT group had the greatest increase in saturation, 2.08 (±0.64) fold increase over baseline. DD exhibited a 1.74 (±0.36) fold increase and ID, a 1.44 (±0.62) fold increase over baseline (FIG. 10). At 1-hour, there is a statistically significant difference between burn groups ID and DD, p<0.05, groups DD and FT, p<0.05, and groups ID and FT, p<0.001. The only other statistically significant time point for oxygen saturation characteristics was at 72 hours, between ID and DD (p<0.05) and DD and FT (p<0.01).

### Assessing Early Differences Between Burn Groups

Intermediate Dermal (ID) versus Deep Dermal (DD). Intermediate versus deep dermal perfusion measurements were statistically different at 1 hour for the following parameters: deoxyHb, (p<0.001); tHb, (p<0.01); and oxygen saturation (StO₂), (p<0.05). oxyHb did not differentiate intermediate versus deep dermal burns at 1 hour or 24 hours, p=0.95 and p=0.41, respectively.

Deep Dermal (DD) versus Full-Thickness (FT). Deep dermal versus full-thickness injury perfusion measurements were statistically different at 1 hour for the following parameters: deoxyHb, (p<0.001); tHb, (p<0.001); and StO₂, (p<0.05). oxyHb was not significant at 1 hour (p=0.09). At 24, 48, and 72-hour time points, all parameters remained statistically significant, with the exception of saturation.

Intermediate Dermal (ID) versus Full-Thickness (FT). Intermediate dermal versus full-thickness burns were statistically different for the following parameters at 1 hour: deoxyHb, p<0.001; tHb, p<0.001; and StO₂, p<0.001. OxyHb was not statistically significant at 1 hour, with p=0.14. As for all other comparisons, oxygen saturation was only significant at 1 hour and 72 hours, while all other parameters remained significant for the rest of the time points: 24, 48, and 72 hours.

### Early Prediction of Burn Depth

When 1-hour tissue oxygen saturation was related to final 72 hour burn depth, there was a strong non-linear correlation (R²=0.99) between StO₂ and burn depth. This suggested that early 1 hour tissue oxygen saturation may be related to final burn depth three days later (FIG. 11).

Perfusion Profiles

Intermediate dermal (ID) burn group exhibited the greatest increase in oxyHb, deoxyHb, and tHb throughout the study, followed by DD burns, which also demonstrated an increase in perfusion parameters, albeit to a lesser degree. FT burns exhibited a decrease in perfusion parameters (FIGS. 7-9). As part of the dermis is destroyed, an inflammatory reaction takes place, which signals for increased perfusion to the burn site. The more superficial the burn, the more of the dermal microvasculature is intact, and thus an increased demand in perfusion can be met with an increased supply and delivery to tissues. In deeper burns, where more of the dermal microvasculature is destroyed, perfusion is compromised.

Another finding in FT injury was increased oxyHb to 1.49 over baseline at 1 hour, before its drop at 24 hours. Possible explanations include the formation of traumatic microhemorrhages due to extreme thermal damage, poor oxygen extraction by the severely injured tissue, or, a combination of both.

### Oxygenation Profiles

An interesting finding that did not follow prior NIR results was tissue oxygenation at 1-hour post injury. (Sowa, et al. 2001 Burns 27(3):241-249; Cross, et al. 2007 Wound Repair and Regeneration 15(3):332-340.) FT burns exhibited the greatest increase in StO₂, 2.08 fold over baseline; DD burns exhibited a 1.74 fold increase and ID burns, the smallest increase over baseline: 1.44 (FIG. 10). This observation may be due acute changes in vascular permeability after injury with vessels sustaining more damage and exhibiting greater vascular leak

### Differentiating Burn Depth

It was found that the best predictive differentiators of burn depth at the majority of time points were tHb and deoxyHb, with the trend being: greatest rise in perfusion signifies more superficial injury. OxyHb was quite variable and did not reliably differentiate burn depths until 48 hours. StO₂ was statistically significant at 1 hour between all the burn depths, but this difference disappeared at 24 and 48 hours, suggesting oxygen saturation change at the depth measured with vsHSI, is transient. This early change in oxygen saturation is highly correlated to final burn depth and may be a predictive indicator of burn severity.

In this specification and the appended claims, the singular forms “a,” “an,” and “the” include plural reference, unless the context clearly dictates otherwise.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

Equivalents

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

1. A method for predicting a maximal depth of thermal burn injury formation in superficial tissue of a subject, comprising:
acquiring photographic imagery of one or more areas of superficial tissue of the subject at one or more wavelengths of light and one or more time points; and
characterizing the obtained photographic imagery to measure one or more physiological properties in the one or more areas of superficial tissue to predict the maximal depth of burn injury formation in superficial tissue of the subject.

2. The method of claim 1, wherein the one or more physiological properties are selected from tissue oxygenation and perfusion levels after exposure to thermal injury.

3. The method of claim 2, wherein the one or more wavelengths of light are selected from the range from about 350 nm to 1,200 nm.

4. The method of claim 1, wherein the photographic imagery is obtained within from about 1 hour to about 48 hours after a thermal exposure to predict the maximum burn depth.

5. The method of claim 1, wherein measuring one or more physiological properties comprises detecting and quantifying the level of oxygenated hemoglobin and an increase or decrease in measured levels of oxygenated hemoglobin in the burned area of the subject is used as a biomarker to predict maximum burn depth.

6. The method of claim 1, wherein measuring one or more physiological properties comprises detecting and quantifying the level of de-oxygenated hemoglobin and an increase or decrease in measured levels of de-oxygenated hemoglobin in burned area of a subject is used as a biomarker to predict maximum burn depth.

7. The method of claim 1, wherein measuring one or more physiological properties comprises detecting and quantifying the level of tissue oxygen saturation and an increase or decrease in measured levels of tissue oxygen saturation in the burned area of a subject is used as a biomarker to predict maximum burn depth.

8. The method of claim 1, wherein measuring one or more physiological properties comprises detecting and quantifying the level of total hemoglobin and an increase or decrease in measured levels of total hemoglobin in the burned area of a subject is used as a biomarker to predict maximum burn depth.

9. The method of claim 1, wherein characterizing the obtained photographic imagery is performed in conjunction with assessment of one or more of the following: collagen, lipids, water, melanin, or other naturally occurring molecules.

10. The method of claim 1, further comprising:
characterizing the obtained photographic imagery to measure one or more physiological properties in the one or more areas of superficial tissue to estimate total body surface area of a patient's burned skin.

11. A biomedical imaging method for predicting acute skin reactions after exposure to ionizing radiation, comprising:
acquiring photographic imagery of one or more areas of superficial tissue of the subject at one or more wavelengths of light and one or more time points; and
characterizing the obtained photographic imagery to detect changes in tissue oxygenation and perfusion levels of the subject to predict acute skin reactions.

12-19. (canceled)

20. A biomedical imaging method for predicting acute skin reactions after exposure to thermal injury, comprising:
acquiring photographic imagery of one or more areas of superficial tissue of the subject at one or more wavelengths of light and one or more time points; and
characterizing the obtained photographic imagery to detect changes in tissue oxygenation and perfusion levels of the subject to predict acute skin reactions.

21-24. (canceled)

25. The method of claim 20, wherein measuring one or more physiological properties comprises detecting and quantifying oxygenated hemoglobin levels and an increase or decrease in measured levels of oxygenated hemoglobin in the exposed area of a subject is used as a biomarker to predict acute skin reaction.

26. The method of claim 20, wherein measuring one or more physiological properties comprises detecting and quantifying de-oxygenated hemoglobin levels and an increase or decrease in measured levels of de-oxygenated hemoglobin in the exposed area of a subject is used as a biomarker to predict acute skin reaction.

27. The method of claim 20, wherein one or more physiological properties comprises detecting and quantifying the level of tissue oxygen saturation and an increase or decrease in measured levels of tissue oxygen saturation in the burned area of a subject is used as a biomarker to indicate predict acute skin reaction.

28. The method of claim 20, wherein one or more physiological properties comprises detecting and quantifying the level of total hemoglobin and an increase or decrease in measured levels of total hemoglobin in the burned area of a subject is used as a biomarker to predict acute skin reaction.

29. The method of claim 20, wherein characterizing the obtained photographic imagery is performed in conjunction with assessment of one or more of the following: collagen, lipids, water, melanin, or other naturally occurring molecules.

30. The method of claim 1, wherein the subject is a human.

31. The method of claim 1, wherein the subject is an animal.

32. The method of claim 1, wherein a computer algorithm for image processing is used in characterizing the obtained photographic imagery.

33. The method of claim 1, further comprising
determining course of medical treatment or segregation of individual subjects into groups for triage in a mass casualty scenario, and
segregating individual subjects into groups for triage in a mass casualty scenario.

34. (canceled)
